(19) United States
(12) Patent Application Publication
(10) Pub. No.: US 2024/0425794 A1
(43) Pub. Date: Dec. 26, 2024

(54) INCREASING MICROBIAL CONCENTRATION BY pH CYCLING

(71) Applicants: Steven Shoup, New Plymouth, ID (US); Barry Roberts, Sandy Springs, GA (US)

(72) Inventors: Steven Shoup, New Plymouth, ID (US); Barry Roberts, Sandy Springs, GA (US)

(21) Appl. No.: 18/464,633

(22) Filed: Sep. 11, 2023

Related U.S. Application Data
(60) Provisional application No. 63/509,292, filed on Jun. 21, 2023.

(51) Int. Cl. C12M 1/34; C12M 1/00; C12M 1/06

(52) U.S. Cl. CPC C12M 41/26; C12M 27/02; C12M 47/00

(57) ABSTRACT

Compositions and methods are provided for increasing a concentration of microbial isolates in a brew by a pH cycling of the brew's pH values. Specifically, cycling the brew's pH from a starting pH value to lower pH values and increasing the brew's pH by the additions of a basic solution.

### CROSS REFERENCE TO RELATED APPLICATION

This application claims priority to U.S. Provisional Application No. 63/509,292, filed Jun. 21, 2023, which is incorporated herein by reference in its entirety.

### BACKGROUND OF THE INVENTION

Plant growth promoting microorganisms have been demonstrated to enhance plant productivity and increase crop yields. Soil amendments have been formulated that contain microbes. The rhizosphere is the soil region that subject to the influence of plant roots. In naturally fertile soils, the rhizosphere can be home to a rich diversity of microorganisms, many of which benefit plants by suppressing pathogenic invasions and helping the plants to acquire vital nutrients from the soil. These microbes create a defensive barrier around the plant roots significantly reducing soilborne pathogens and plant parasitic nematodes. As well, microorganisms that live on or near the plant root systems can significantly improve nutrient absorption and water retention of the soil. In addition, the microbes activate soil nutrients and releases bound natural fertilizers to produce more nutritious crops and enhance plant growth.

For maximum effectiveness, it is important to have a large concentration of a broad spectrum of the microbial isolates. Rhizobacteria can suppress pathogen ingress into the plant roots and are essential for biological nitrogen fixation. Actinomycetes produce powerful antibiotic compounds and eradicate a large array of soil borne pathogens and nematodes. Other bacterial isolates can accelerate the microbial processes that augment the availability of nutrients in a form easy to assimilate by plants. Similarly, fungi are organisms that can secrete growth promoting hormones. In addition, saprophytic fungi can produce secondary metabolites that are active in suppressing soil borne pathogens and plant parasitic nematodes. Endomycorrhizae live in the root tissue and are powerful growth stimulators. Ectomycorrhizae form a sheath around plant roots preventing nematodes and pathogen penetration as well as promote nutrient absorption. In general, products with higher microbial abundance and diversity have a greater chance of containing microbes that perform a particular beneficial function under a range of conditions.

However, growing multiple different microbes together or having them coexist can be problematic. Many microbes are antagonistic to each other or have faster growth rates at certain conditions monopolizing essential nutrients. Thus, incorporating wide spectrum of microbes is simultaneously as challenging as it is desirable.

Naturally, the greater the concentration of the beneficial microbes, the less of the product is required and thus produces a more economically viable product. In order to create a high concentration of microbes, it is important to have a food source that is extremely nutritious for the broad array different microbes. The media generally needs to contain sources of carbon, nitrogen and vitamins. Dextrose is a widely utilizable carbon source for microbes and is the most commonly used growth media. Nitrogen sources can often include peptone, yeast extract, and malt extract. Again, the problem exists on how to ensure all the different classes of microbes can grow without monopolizing the food source required by the other microbes.

A further problem exists when one type of microbe grows too rapidly. The brew will tend to form a film or a matty layer created by the over-abundance of a particular microbe. These matty layers or matts can be found on the top of the brew, on the bottom, or somewhere in the middle depending on the particular microbe. This matty layer creates a problem during the processing of batch into a marketable soil amendment. These matts are difficult to break down and are difficult to filter out of the brew. Consequently, these matts present an issue for creating a water-soluble product.

Numerous other fields can benefit from similar all-natural organic solutions. Thus, there is continuing need for improved biological solutions. These improved biological solutions need a to contain a high microbial abundance with a diversity of classes of microbes.

### BRIEF SUMMARY OF THE INVENTION

The present invention discloses compositions and methods in various embodiments for increasing a concentration of microbial isolates with a diversity of classes of microbes in a brew by a pH cycling of the brew's pH values.

In one embodiment, disclosed is a method of increasing a concentration of microbial isolates in a brew by a pH cycling of the brew's pH from a starting pH value to a lower pH value and increasing the brew's pH by an addition of a basic solution to a higher pH value. The brew's pH is reduced from the higher pH value to a second lower pH value. The pH cycling prevents any of the microbial isolates from forming a matty layer.

In this embodiment, the brew's pH is again increased from the second lower pH value to a second higher pH value by a second addition of a second basic solution.

In addition, the concentration of the microbial isolates is further increased by the microbial isolates feeding for a period of time increasing the brew's pH to an even higher pH value than the second higher pH value which can be in the 7 s for a pH value.

In this embodiment, the brew's pH can be further cycled, lowering from the second higher pH value to a third lower pH value. The brew's pH is again increased from the third lower pH value to a third higher pH value by a third addition of another basic solution. At least one of the first lower pH value, the second lower pH value, and the third lower pH value is in the 4 s.

Again, the concentration of microbial isolates is further increased by the microbial isolates feeding for a period of time increasing the brew's pH to an even higher pH value than the third higher pH value.

In this embodiment, the first basic solution and the second basic solution can be the same basic solution. The first basic solution and the third basic solution can also be the same basic solution. As disclosed, at least one of the first basic solution, the second basic solution, and the third basic solution is sodium carbonate.

Further disclosed, the microbial isolates comprise of at least 3 classes from the classes of aerobic bacteria, anaerobic bacteria, rhizobacteria, actinomycetes, endomycorrhizae, ectomycorrhizae, and saprophytic fungi.

The brew is agitated by mechanical agitation during the cycling and the temperature of the brew is held relatively constant. Preferably, the temperature is held between above 80 degrees Fahrenheit. In addition, the brew is aerated by an external aeration mechanism. The aeration comprises compressed dried air run through a filter.

Preferably, the brew is lyophilized after increasing the concentration of the microbial isolates.

### BRIEF DESCRIPTION OF THE DRAWINGS

Various embodiments are illustrated by way of examples in the figures of the accompanying drawings. Such embodiments are demonstrative and not intended to be exhaustive or exclusive embodiments of the present subject matter. The drawings are not necessarily to scale, emphasis instead being placed on illustrating the principals of the invention.

FIG. 1 is a flow chart depicting the major steps of the preferred embodiment of the present invention.

FIG. 2 depicts the step of cooking a microbial food source.

FIG. 3 depicts the step of brewing a microbial solution.

FIG. 4 depicts the lyophilizing a microbial medium.

### DETAILED DESCRIPTION OF THE INVENTION

The description that follows includes compositions, systems, methods, and apparatuses that embody various elements of the present disclosure. However, it should be understood that the described disclosure may be practiced in a variety of forms in addition to those described herein. Accordingly, the referenced drawings show, by way of illustration, specific embodiments in which the invention may be practiced. It is to be understood that other embodiments may be utilized, and structural changes may be made without departing from the scope of the claims. It is further understood that the steps described with respect to the disclosed processes may be performed in differing order and are not limited to the order presented herein.

The present invention discloses compositions and methods in various embodiments for increasing a concentration of microbial isolates in a brew by a pH cycling of the brew's pH values. The preferred embodiments are described below.

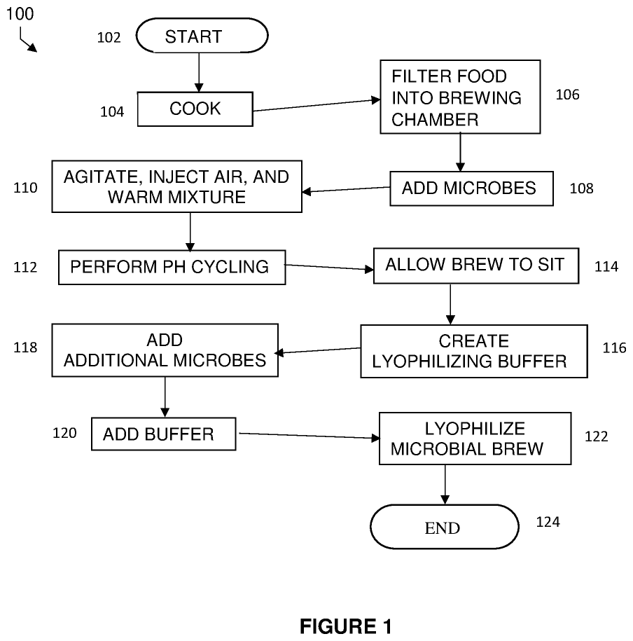

Referring to FIG. 1, illustrated is a flowchart 100 for the major steps of enhancing the concentration in a brew of microbes with a diversity of classes.

Following the start step 102, a food source is cooked as described in step 104.

In step 104, care should be used to choose foods and components that are 100% water soluble and stay in suspension to help ensure water solubility of the final product.

Honey should be considered as a food source since it contains a high concentration of sugars and essential vitamins especially the B vitamins. However, honey has low glass transition temperatures (Tg), which makes freeze drying difficult. During dehydration, honey typically becomes extremely sticky, hard and very difficult to handle as a product. Although honey does not lyophilize well, when used as a food source, the microbial count skyrockets compared to most other food sources.

Start with 50% brewer's yeast by weight and 50% processed honey by weight. The brewer's yeast selected should be 100% water soluble. Processed honey with the waxes removes is used to help ensure solubility. The industry typically uses around 10% yeast; however, a 50% yeast mixture increases the yield when combined with honey. To ensure consistency, the honey should be a blend that uses a large variety of honeys from multiple sources.

In step 106, the food source is filtered and added into a brewing chamber. Cool the food mixture to below 95 degrees. The cooked mixture is then filtered and cleaned down to at least 10 microns before going into a brewing vessel. The filtering removes most of the non-soluble material in the solution including the yeast cell wall debris. The resultant is approximately 39 to 40 liters of food to be added to the brewing vessel.

In step 108, after adding the food mixture, 8 liters of microbes from a previous batch is added to the feeding tank. It is preferred to have a broad spectrum of the microbial isolates. Rhizobacteria can suppress pathogen ingress into the plant roots and are essential for biological nitrogen fixation. Actinomycetes produce powerful antibiotic compounds and eradicate a large array of soil borne pathogens and nematodes. Other aerobic bacteria and anaerobic bacterial isolates can accelerate the microbial processes that augment the availability of nutrients in a form easy to assimilate by plants. Similarly, fungi are organisms that can secrete growth promoting hormones. In addition, saprophytic fungi can produce secondary metabolites that are active in suppressing soil borne pathogens and plant parasitic nematodes. Endomycorrhizae live in the root tissue and are powerful growth stimulators. Ectomycorrhizae form a sheath around plant roots preventing nematodes and pathogen penetration as well as promote nutrient absorption. By using the disclosed pH cycling, the concentration of these microbes is greatly increased and are brewed together.

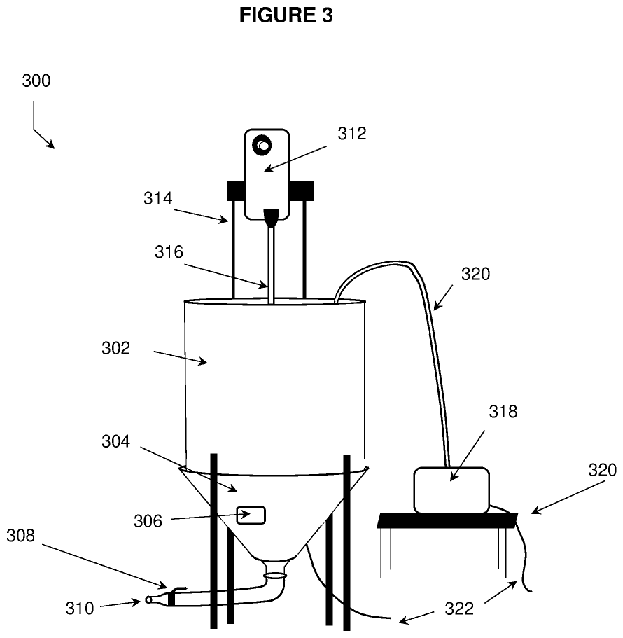

The step of brewing 110 of the microbial solution is depicted in reference to FIG. 3. In step 110, agitation and air injection at bottom is started after adding the microbes. Compressed and dried filtered air is injected into the feeding tank. Direct oxygen can be detrimental. A light bubble similar to a fish tank is utilized just to keep brew oxygenated. The stirring rotates at 45 to 70 rpm to keep the mixture moving and lightly agitated. External heating pads surrounding the vessel are set to keep the mixture to around 85-95 degrees.

In step 112, after adding the microbes, the resultant mixture is around 6.3 pH. In order to maximize the growth of a wide diversity of microbes with the temperature held constant, the pH needs to be managed such that all the microbes grow without any one microbe severely dominating. Accordingly, the brewing during the growing process is controlled to a pH of around 6 to a lower pH in the mid 4 s. This cycle allows the different groups of microbes to multiply. Since different microbes thrive at differing pHs, cycling the pH ranges allows the microbes to grow together to feed and multiply without other microbes dominating. If the brew is allowed to sit too long in the low 4s pH, a resultant acidophilic actinomycetes bloom occurs. The cycling of the pHs allows the microbes to grow without any one particular microbe dominating and eliminates the matting layer issue.

Mined sodium carbonate that is completely water soluble is preferred to be used to alter the pH. Use 5% mixture of sodium carbonate to water. When the brew is in the mid 4 s pH, add sodium carbonate while continuing to agitate. The first pH crash to mid 4 s pH usually takes around 5 to 5.5 hours and will need about 2 liters of the sodium carbonate to raise the batch to around a 6 pH. The second crash to the mid 4 s PH usually takes less time of 3 to 4 hours and the addition of around 3 liters to raise the pH back to around a pH of 6. The third crash may take as long as 13 hours. Typically, the total of 8.5 to 9 liters of the sodium carbonate is used for the 3 pH cycling runs.

Running the brew through two pH cycles greatly increases the microbial yields of all the differing microbes. A third cycle is preferred to be performed to further increase microbial count. At the end of the pH brew cycling, the brew will be at approximately a 6 ph.

In step 114, allow the brew to sit for another 48 hours in order to maximize count. As the brew sits, the brew will creep up into 7 s pH. Some of microbes will diminish if the brew hits an 8 pH.

In step 116, the lyophilizing buffer or excipient is created. In a large mix vessel, add 3200 grams of cane sugar to water until the total volume is 4 liters. Mix until the cane sugar is fully dissolved. Ideally, the mixture is concentrated to act as a buffer with the cane sugar staying suspended at room temperature. In order to make the product rise as a puff cake, an 8% mixture is used. Over a 10% concentration will start killing microbes as the sucrose is toxic to some of the microbes. Under 5%, the end product is sticky and gooey and is difficult to create a water-soluble product.

In step 118, any additional microbes are added to the microbial brew. If endo-mycorrhizae are to be added to brew, it should be added at this time. Endo-mycorrhizae may take up to 10 days to brew. Most endo-mycorrhizae are grown on plant roots and harvested by scraping or crushing the roots. Endo-mycorrhizae grown this way make the product non-water soluble. However, certain foods can allow endo-mycorrhizae to grow using the methodology described in this invention and can be water soluble.

In step 120, add the sugar solution to brew containing the microbes. Turn the agitator up and slowly add the sugar buffer solution. The lyoprotectant used in this embodiment of the invention is sucrose, which is a glucose linked via its C1 carbon to fructose. By using honey and sucrose as a dehydration buffer, the final product becomes a fluffy cake with a fine texture and excellent solubility characteristics. If there is not enough sugar, the end product is a sticky mass that clumps because of honey and resists becoming soluble in water.

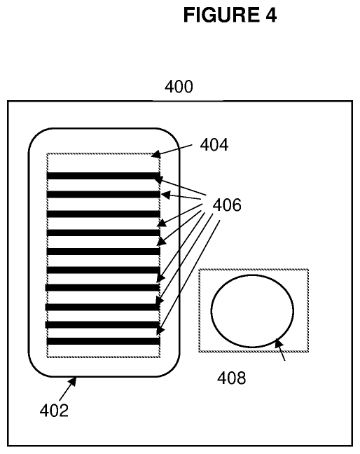

In step 122, the microbial medium is lyophilized as depicted in reference to FIG. 4. Pour the buffered solution into trays for lyophilization. In this preferred embodiment, 2100 ml is poured into a tray and then set programming. Lyophilize the solution for approximately 60 hours to 70 hours. Preserving microbes by lyophilization necessitates that the microbes are suspended in a medium that helps to maintain their viability through freezing, water removal, and subsequent storage. The ideal solution will have a component that helps to form a solid “cake” which gives body to the bacterial suspension once freeze dried.

In step 124, after lyophilizing, the microbial medium is ready for any post lyophilizing processing and the process ends.

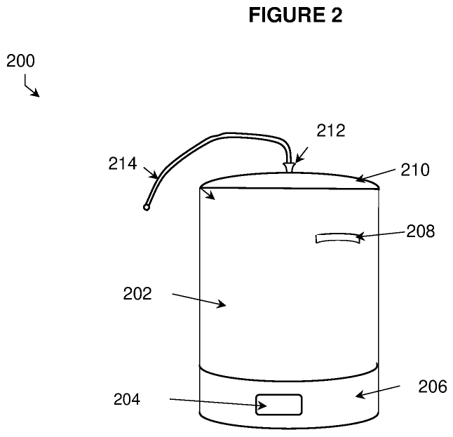

Referring to FIG. 2, the step of cooking 104 the microbial food source is depicted. In the preferred embodiment, 1000 grams of honey is mixed with 1000 grams of brewer's yeast in a commercial cooker 200. The commercial cooker 200 has a cooking chamber 202 with a handle 208, a lid 210, a relief valve 212 with attached tubing 214, and a heating element casing 206 with a heater control panel 204. Water is added until there is 42 liters of mixture is in the cooker. The mixture is cooked at a simmer for 20 minutes. This cooking kills any live unwanted bacteria in the water and breaks open the cells of the yeast to create a yeast extract. The cooking temperature should not be hot enough to denature the proteins.

Referring to FIG. 3, depicted are the steps of brewing 108-114 of the microbial solution in a brewing vessel assembly 300. The brewing vessel 302 is an insulated stainless steel cone bottom tank. A nozzle valve 308 at the bottom of the brewing assembly 302 enables the solution to be withdrawn after brewing via the exit nozzle 310.

Agitation is provided by an agitator rotation rod 316 supported by the agitator support stand 314. The agitator controller 312 rotates the agitator rotation rod 316 speed at 45 to 70 rpm to keep the mixture moving and lightly agitated.

Air injection at bottom of the brewing vessel 302 is started after adding the microbes. Compressed and dried air is run through medical grade air filtration system 318 before injection into the feeding tank 302 by an air tube 320. Direct oxygen can be detrimental. A light bubble similar to a fish tank is utilized just to keep brew oxygenated. Power is supplied to the air filtration system 318 by a power cord 322.

A heating control panel 306 governs the external heating pad 304 that surround the bottom conical section of the vessel 302. The heating pad 304 is set to 90° F. warm, which should keep the mixture to around 85-95 degrees. Power to the vessel assembly 300 is supplied via the power cords 322.

In order to maximize the growth of a wide diversity of microbes with the temperature held constant, the pH needs to be managed such that all the microbes grow without any one microbe severely dominating. Running the brew through two pH cycles greatly increases the microbial yields of all the differing microbes. A third cycle is preferred to be performed to further increase microbial count.

Referring to FIG. 4, depicted is the step of the lyophilizing 122 the microbial medium. The microbial medium is poured into trays 406 and placed on the shelves 404 in the vacuum chamber 402 of the lyophilizer 400 for dehydration. A motor (not shown) is in place to ensure the vacuum chamber 402 is vacuum-tight when closed. The condenser 408 consists of refrigerated coils or plates. During the drying process, ice moves from the frozen product to the condenser by sublimation. Preferably, the final product becomes a fluffy cake with a fine texture and excellent solubility characteristics.

1. A method of increasing a concentration of microbial isolates in a brew by a pH cycling of the brew's pH from a starting pH value to a first lower pH value and increasing the brew's pH by a first addition of a first basic solution to a first higher pH value, and the brew's pH is reduced from the first higher pH value to a second lower pH value.

2. The method of claim 1, wherein the brew's pH is increased from the second lower pH value to a second higher pH value by a second addition of a second basic solution.

3. The method of claim 2, wherein the concentration of the microbial isolates is further increased by the microbial isolates feeding for a period of time increasing the brew's pH to an even higher pH value than the second higher pH value.

4. The method of claim 2, wherein the brew's pH is lowed from the second higher pH value to a third lower pH value.

5. The method of claim 4, wherein the brew's pH is increased from the third lower pH value to a third higher pH value by a third addition of a third basic solution.

6. The method of claim 5, wherein the concentration of microbial isolates is further increased by the microbial isolates feeding for a period of time increasing the brew's pH to an even higher pH value than the third higher pH value.

7. The method of claim 2, wherein the first basic solution and the second basic solution are the same basic solution.

8. The method of claim 5, wherein the first basic solution and the third basic solution are the same basic solution.

9. The method of claim 5, wherein at least one of the first basic solution, the second basic solution, and the third basic solution is sodium carbonate.

10. The method of claim 1, where the microbial isolates comprise of at least 3 classes from the classes of aerobic bacteria, anaerobic bacteria, rhizobacteria, actinomycetes, endomycorrhizae, ectomycorrhizae, and saprophytic fungi.

11. The method of claim 1 wherein the brew is agitated by mechanical agitation during the cycling.

12. The method of claim 1 wherein a temperature of the brew is held relatively constant.

13. The method of claim 12, the temperature is held between 84 and 91 degrees Fahrenheit.

14. The method of claim 1, wherein the brew is aerated by an external aeration mechanism.

15. The method of claim 14, wherein aeration comprises dried air run through a filter.

16. The method of claim 4, wherein at least one of the first lower pH value, the second lower pH value, and the third lower pH value is in the 4 s.

17. The method of claim 6, wherein the even higher pH value is in the 7 s.

18. The method of claim 5, wherein at least one of the first higher pH value, the second higher pH value, and the third higher pH value is below the starting pH value.

19. The method of claim 1, wherein the pH cycling prevents any of the microbial isolates from forming a matty layer.

20. The method of claim 1, wherein the brew is lyophilized after increasing the concentration of the microbial isolates.
